(19) United States
(12) Patent Application Publication
(10) Pub. No.: US 2021/0108227 A1
(43) Pub. Date: Apr. 15, 2021

(54) Parvovirus Vector Production

(71) Applicant: GlaxoSmithKline Intellectual Property Development Limited, Brentford Middlesex (GB)

(72) Inventor: Christopher HERRING, Stevenage (GB)

(21) Appl. No.: 17/047,730

(22) PCT Filed: Apr. 16, 2019
(86) PCT No.: PCT/EP2019/059797

§ 371 Date: Oct. 15, 2020

(30) Foreign Application Priority Data
Apr. 18, 2018 (GB) 1806333.9

(51) Int. Cl. C12N 15/86; C12N 7/02; C07K 14/245

(52) U.S. Cl. CPC C12N 15/86; C12N 7/02; C12N 2750/14152; C12N 2750/14143; C07K 14/245

(57) ABSTRACT

Provided is a cell containing a nucleic acid sequence having a parvovirus terminal repeat sequence, wherein the cell overexpresses single strand binding protein compared to a cell of a wild-type (WT) strain of the same species. Also provided is a nucleic acid vector containing a nucleic acid sequence having a parvovirus terminal repeat sequence and a nucleic acid sequence encoding a single strand binding protein. Methods of using said nucleic acid vector to propagate and purify nucleic acid vectors involved in the production of parvovirus vector particle production are also described.

### FIELD OF THE INVENTION

The invention relates to nucleic acid vectors comprising nucleic acid sequences required for parvovirus vector particle production, and uses thereof. Also provided are methods of propagating and purifying the nucleic acid vectors described herein used in recombinant parvoviral vector production.

### BACKGROUND TO THE INVENTION

Viral vector systems have been proposed as an effective gene delivery method for use in gene therapy (Verma and Somia (1997) Nature 389: 239-242). More recently, parvoviruses of the Parvovirinae family, such as the dependoparvovirus, Adeno-Associated Virus (AAV), the bocaparvovirus, Human Bocavirus (HBoV) and even an AAV vector pseudotyped with an HBoV capsid (Yan et al. (2013) Mol Thera, 21: 2181-2194) have been identified as desirable viral vectors for gene therapy applications.

The genome of a parvovirus consists of a linear single stranded DNA genome with terminal repeat sequences at each end. These terminal repeats contain palindromic sequences which give rise to secondary structures, such as hairpins and cruciforms that are essential for replication initiating second strand DNA synthesis (Shen et al. (2016) J Virol 90:7761-7777). Furthermore, the palindromic terminal repeat sequences and their secondary structures are essential for packaging recombinant DNA genome of the parvovirus into the parvovirus virion (McLaughlin et al., (1988), J Virol 62:1963-1973; Samulski et al., (1989), J Virol 63: 3822-3828; Balague et al., 1997, J Virol 71:3299-3306).

For production of recombinant parvovirus vector particles used in gene therapy, the native genome of the parvovirus is modified to remove the genes between the terminal repeat sequences encoding the regulatory and structural proteins and replaced with a gene of interest (transgene).

Thus, the recombinant DNA genome of the parvovirus comprises a transgene flanked by the terminal repeat sequences. Plasmids comprising nucleic acid sequences of the recombinant DNA genome of the parvovirus are commonly known as a transfer vector or a transfer plasmid. The parvovirus genes encoding the regulatory and structural genes removed from the native parvovirus DNA genome are provided in trans, along with genes derived from a helper virus (helper genes) if the recombinant parvovirus to be produced is a dependoparvovirus.

During the processes of cloning and propagation of the recombinant DNA genome (transfer vector) for recombinant vector particle production, stability of the terminal repeat sequences has been found to be problematic, leading to deletions of nucleotides within the terminal repeat sequences or the entire terminal repeat sequence at one or both termini flanking the transgene. This is a known problem in the field, which, for example, has been reported by the Viral Vector Facility, Zurich, (“Widespread deletion of 11 bp within one ITR of AAV-2 vector plasmids”, Viral Vector Facility, Zurich) and by Petri et al. (Petri et al, (2014) BioTechniques 56:269-273) and poses a significant challenge to the efficiency of manufacture of recombinant parvovirus vector particles.

It is postulated that the secondary structure resulting from the palindromic nature of the terminal repeat sequences are acted on by various cellular mechanisms, thereby contributing to the instability of the terminal repeat sequences. Examples of cellular mechanisms include replication, recombination, DNA repair and nucleases specifically targeting secondary structures (Connelly and Leach, (1996) Genes to Cells, 1:285-291; Darmon et al, (2010) Mol Cell 39:59-70; Bikard et al, (2010) Microbiol Mol Biol Rev 74:570-588)

Despite current methods to try and alleviate the problem, such as using recombination protein RecA and/or hairpin nuclease SbcCD protein deficient E. coli strains (Darmon et al., (2010) Mol Cell 39:59-70; Agilent Technologies, “AAV Helper-Free System” Instruction Manual), instability of parvovirus terminal repeat sequences during cloning and propagation remains an issue, particularly for scale up of manufacture during initial cloning steps and scale up.

It is therefore an object of the present invention to provide a nucleic acid vector, uses thereof and a method of propagating nucleic acid vector described herein used in recombinant parvovirus vector particle production, for improving the stability of the parvovirus terminal repeats.

### SUMMARY OF THE INVENTION

The inventors have surprisingly found that improved stability of the parvovirus terminal repeat sequences is achieved when the terminal repeat sequences are manipulated, for example, cloned or propagated, in prokaryotic cells overexpressing single strand binding (SSB) protein.

Therefore, according to one aspect of the invention, there is provided a prokaryotic cell comprising a nucleic acid sequence comprising a parvovirus terminal repeat sequence, wherein the prokaryotic cell overexpresses single strand binding protein compared to a prokaryotic cell of a wild-type (WT) strain of the same species.

According to a further aspect of the invention, there is provided a nucleic acid vector comprising a nucleic acid sequence comprising a parvovirus terminal repeat sequence and a nucleic acid sequence encoding a single strand binding protein.

According to a further aspect of the invention, there is provided a use of the nucleic acid vector as described herein in the production of a recombinant parvovirus vector particle. The recombinant parvovirus vector particle may be a recombinant AAV vector particle or recombinant BoV vector particle.

According to yet another aspect of the invention, there is provided a method of propagation and purification of a nucleic acid vector described herein comprising the steps of:

(i) introducing a nucleic acid vector as described herein into a cell

(ii) growing a culture of the cell of step (i)

(iii) harvesting and lysing the cells of step (ii)

(iv) purifying plasmid DNA from the lysed cells of step (iii).

### DESCRIPTION OF DRAWINGS/FIGURES

FIG. 1: An agarose gel showing SmaI digests of transfer vector plasmids

FIG. 2: An agarose gel showing SmaI digests of transfer vector plasmids at 30° C. and 37° C.

### DETAILED DESCRIPTION OF THE INVENTION

### Definitions

Unless defined otherwise, all technical and scientific terms used herein have the same meaning as is commonly understood by one of skill in the art to which this invention belongs. All patents and publications referred to herein are incorporated by reference in their entirety.

The term “comprising” encompasses “including” or “consisting” e.g. a composition “comprising” X may consist exclusively of X or may include something additional e.g. X+Y.

The term “consisting essentially of” limits the scope of the feature to the specified materials or steps and those that do not materially affect the basic characteristic(s) of the claimed feature.

The term “consisting of” excludes the presence of any additional component(s).

The term “terminal repeat sequence” refers to the palindromic sequences at the termini of the parvoviral genomic DNA, which form secondary structures such as hairpins and cruciforms, and are necessary for replication of the genomic DNA or recombinant genomic DNA. The terminal repeat sequences of parvoviruses are well known in the art and well characterised to be essential for replication, packaging and integration events of the parvovirus (e.g. Shen et al, (2016) J Virol 90:7761-7777).

The term “nucleic acid vector” refers to a vehicle which is able to artificially carry foreign (i.e. exogenous) genetic material into another cell, where it can be replicated and/or expressed. Examples of nucleic acid vectors include but are not limited to plasmids, minicircles, bacterial artificial chromosomes (BACs), yeast artificial chromosomes (YACs), P1-derived artificial chromosomes (PACs), cosmids or fosmids.

The term “nucleic acid sequence” within the context of a nucleic acid vector refers to the DNA of the nucleic acid vector. Nucleic acid sequences may comprise genetic elements such as terminal repeats, promoters or a transcription terminator, or may encode proteins.

The term “vector particle” or a “virion” in the context of a parvovirus, refers to a parvovirus capsid particle suitable for carrying a DNA genome of the parvovirus, which in the case of a recombinant parvovirus vector particle will comprise of a transgene flanked at either end by a parvovirus terminal repeat sequence. The terms “vector particle” and “viral vector” are used interchangeably.

Where the “vector particle”, “virion”, “DNA genome of the parvovirus” or “genetic material” is described as being “recombinant” herein, it is meant that the wild-type version of the DNA or parvovirus vector particle has been modified, generally by inclusion DNA from a different source. Therefore, a recombinant parvovirus vector particle, or recombinant DNA genome of the parvovirus, will have nucleic acid sequences from a different source, generally a gene of interest.

The terms “transformation” and “transduction” as used herein, may be used to describe the insertion of the nucleic acid vector or viral vector into a target cell. Insertion of a nucleic acid vector is usually called transformation for bacterial cells, although insertion of a viral vector may also be called transduction. The skilled person will be aware of the different non-viral transfection methods commonly used, which include, but are not limited to, the use of physical methods (e.g. electroporation, cell squeezing, sonoporation, optical transfection, protoplast fusion, impalefection, magnetofection, gene gun or particle bombardment), chemical reagents (e.g. calcium phosphate, highly branched organic compounds or cationic polymers) or cationic lipids (e.g. lipofection). Many transfection methods require the contact of solutions of plasmid DNA to the cells, which are then grown and selected for a marker gene expression.

The term “functional homologue” is well known in the art and as used herein refers to equivalent proteins (protein homologues) between species or kingdoms. For example, a functional variant of the RecA protein refers to the variants of RecA proteins between bacterial strains.

The term “native promoter” is well known in the art and is used to mean a promoter that drives transcription of a specific gene in a wild-type cell.

### Parovirus

Parvoviruses are subdivided into three major groups, namely densoviruses, autonomous parvoviruses (APV), such as Bocavirus (BoV), and dependoviruses, such as AAV. Densoviruses only infect insets. APV and dependoviruses infect vertebrate animals. Whilst APVs are able to replicate in the target cells without the need of helper viruses, dependoviruses require helper viruses for replication.

The genome of parvoviruses is made up of approximately 5 kilobases (kb) of single stranded DNA. At both ends, or termini, of the genome are sequences known as terminal repeats, which do not encode any protein. In parvoviruses, the terminal repeat sequences are palindromic.

The genome of parvoviruses can be broadly divided into left and right halves, which encode regulatory and structural (capsid) proteins, respectively. The regulatory protein involved in replication is known as Rep or NS (for non-structural protein) and the structural capsid protein is referred to as VP (Ponnazhagan (2004) Expert Opin Biol Ther 4:53-64).

The palindromic terminal repeats form hairpin-like or cruciform structures and are essential for viral genome replication. The terminal repeats are also essential for genome packaging into the viral virion and also for integration of the parvovirus DNA genome into the host chromosome.

Homotelomeric parvoviruses, such as AAV, comprise two genomic terminal repeat sequences that are inverted in sequence and identical in structure. The replication process with homotelomeric parvoviruses are symmetrical. Heterotelomeric parvoviruses, such as HBoV, comprise two genomic terminal repeat sequences that are dissimilar, such that the 3′ terminal hairpin is different to the 5′ terminal hairpin. In HBoV1, the 3′ terminal hairpin forms a rabbit ear structure of 140 nt with mismatched nucleotides, whilst the 5′ terminal hairpin consists of a perfect palindromic sequence of 200 nt in length (Shen et al., (2016) J Virol 90:7761-7777). In other heterotelomeric parvoviruses, namely the minute virus of mice (MVM) and bovine parvovirus (BPV), their 5′ terminal repeat sequences are able to form a cruciform structure. The replication of origin within either hairpin end of both homotelomeric and heterotelomeric parvoviruses contain binding elements for binding by regulatory proteins Rep78/68 or NS1 in AAV or HBoV, respectively.

### AAV

AAV has a linear single-stranded DNA (ssDNA) genome of approximately 4.7-kilobases (kb), with two 145 nucleotide-long inverted terminal repeats (ITR) at the termini for AAV2. The ITRs flank the two viral genes—rep (replication) and cap (capsid), encoding non-structural and structural proteins, respectively, and are essential for packaging of the AAV genome into the capsid and for initiating second strand DNA synthesis upon infection. AAV has been classified as a Dependoparvovirus (a genus in the Parvoviridae family) because it requires co-infection with helper viruses such as adenovirus, herpes simplex virus (HSV) or vaccinia virus for productive infection in cell culture (Atchison et al. (1965) Science 149:754; Buller et al. (1981) J. Virol. 40: 241).

The AAV2 ITR sequences comprise 145 bases each and are the only cis-acting elements necessary for AAV genome replication and packaging into the capsid. Typically, the ITRs will be at the 5′ and 3′ ends of the vector genome and flank the heterologous nucleic acid (transgene), but need not be contiguous thereto. The ITRs are imperfect palindromes with a GC content of 70% that fold back on themselves to form hairpin-like secondary structures (Henckaerts and Linden (2010) Future Virol 5:555-574). The 145 nt sequence contain all of the cis-acting signals needed to support DNA replication, packaging and integration (Mclaughlin et al., (1988) J Virol 62:1963-1973; Samulski et al., (1989) J Virol 63:3822-3828). The ITRs can be the same or different from each other in sequence.

An AAV ITR may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered. An AAV ITR need not have the native terminal repeat sequence (e.g. a native AAV ITR sequence may be altered by insertion, deletion, truncation and/or missense mutations), as long as the terminal repeat mediates the desired functions, e.g., replication, virus packaging, and/or integration, and the like.

The genomic sequences of various native ITRs are well known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching AAV nucleic acid and amino acid sequences.

### BoV

Species within the Bocaparvovirus genus within the Parvoviridae family include Human Bocavirus (HBoV), minute virus of canines (MVC), bovine parvovirus (BPV), porcine bocavirus and gorilla bocavirus.

Bocaviruses are unique from other parvoviruses in that they express a small nuclear phosphoprotein NP1 from an open reading frame located in the middle of the genome. NP1 is a non-structural protein and required for Bocavirus DNA replication (Shen et al. (2016) J Virol 90:7761-7777).

The complete genome of the human bocavirus 1 (HBoV1) may be obtained from GenBank accession no. JQ923422.

### Production of Recombinant Parvoviruses

Methods of recombinant parvovirus vector particle production are well known in the art. In a typical method, a gene of interest (transgene) is cloned between the terminal repeat sequences of the parvoviral genome. A plasmid carrying nucleic acid sequences of the transgene flanked by parvovirus terminal repeat sequences is commonly referred to as a transfer vector. The genes encoding NS/Rep and VP of the wild-type virus, and that of the helper virus proteins as required, are provided in trans. Providing the viral genes in trans ensures that the recombinant parvovirus vector particle produced is replication defective. Accordingly, the transfer vector plasmid, NS/Rep and VP plasmid, and helper plasmid as required, are prepared, propagated and purified at scale in prokaryotic cells before being transfected into mammalian cells. When producing viral vectors for gene therapy, the plasmids as well as the final viral vector particles must adhere to strict practices and regulatory standards (e.g. Good Manufacturing Practice). The transfected cells are then grown, lysed and the recombinant parvoviruses subjected to gradient centrifugation or ion exchange chromatography to purify the recombinant virion particles produced by the mammalian cells.

### Cells

In one aspect of the invention, there is provided a prokaryotic cell comprising a nucleic acid sequence comprising a parvovirus terminal repeat sequence, wherein the prokaryotic cell overexpresses single strand binding protein compared to a cell of a wild-type (WT) strain of the same species. Parvovirus terminal repeat sequences are well known in the art. For example, the terminal repeat sequences of at least AAV and HBoV1 may be obtained from the respective GenBank accession numbers provided above.

In one embodiment, the prokaryotic cell comprises nucleic acid sequences comprising two parvovirus terminal repeat sequences.

In one embodiment, the prokaryotic cell is a bacterial cell. In a further embodiment, the bacterial cell is of the genus Escherichia, Bacillus, Pseudomonas, Streptomyces, Streptococcus or Vibrio. In a preferred embodiment, the cell is an E. coli cell.

Single strand binding (SSB) proteins are well known in the art and are a class of proteins that have been identified and characterised across species in both prokaryotes and eukaryotes, as well as viruses. The function of SSB protein is to bind to single stranded DNA and prevent annealing of single stranded DNA into double stranded DNA and to prevent single strand DNA from degradation. SSB proteins in bacteria are known to be play a role in DNA replication, repair and recombination (Meyer and Laine, (1990) Microbiol Rev 54:342-380). In one embodiment the SSB protein is the variant native to the prokaryotic cell. In one embodiment, the SSB protein is an E. coli SSB protein. The nucleic acid sequence of the E. coli ssb gene may be obtained from GenBank accession no. J01704.

In one embodiment, the prokaryotic cell is a RecA deficient strain. In another embodiment, the prokaryotic cell is a strain deficient for a functional homologue of RecA. RecA is a protein essential for repair and maintenance of DNA, with a central role in homologous recombination. RecA protein functional homologues are well known in the art. For example, the functional homologue in eukaryotes is RAD51 and in archaea is RadA.

In one embodiment, the prokayrotic cell is an SbcCD deficient strain. In another embodiment, the prokaryotic cell is deficient for a functional homologue of the SbcCD protein. The SbcCD protein is a nuclease found prokaryotes and eukaryotes. In E. coli, the SbcCD protein forms a large complex that functions as an ATP-dependent double strand DNA exonuclease and an ATP-independent single strand DNA endonuclease. SbcCD functional homologues are well known in the art.

In one embodiment the parvovirus is an adeno-associated virus (AAV), a Bocavirus (BoV) or a minute virus of mice (MVM).

In one embodiment, the overexpressed SSB protein is a variant endogenous to the WT strain of the prokaryotic cell.

In one embodiment, the cell comprises an exogenous nucleic acid sequence encoding the SSB protein. In one embodiment, the exogenous nucleic acid sequence encodes an SSB protein that is a variant endogenous to the prokaryotic cell.

### Nucleic Acid Vector

According to one aspect of the invention, there is provided a nucleic acid vector comprising a nucleic acid sequence comprising a parvovirus terminal repeat sequence and a nucleic acid sequence encoding a single strand binding protein.

The parvovirus terminal repeat sequence need not be the terminal repeat sequence native to the WT parvovirus (e.g. a native AAV ITR sequence may be altered by insertion, deletion, truncation and/or missense mutations), as long as the terminal repeat mediates the desired functions, e.g., replication, virus packaging, and/or integration, and the like.

In one embodiment, the nucleic acid vector comprises nucleic acid sequences comprising two parvovirus terminal repeat sequences.

In one embodiment, the parvovirus is AAV, BoV or MVM.

In one embodiment the nucleic acid sequence comprising a parvovirus terminal repeat sequence is derived from AAV1, AAV2, AAV3, AAV4, AAV5, AAV6, AAV7, AAV8, AAV9, AAV10, AAV11, AAV12, AAV13 or combinations thereof.

In a further embodiment, the SSB protein is operably linked to a promoter. The promoter is optionally a native promoter of the ssb gene. That is to say, a native promoter of the ssb gene in the WT strain of the same species of the cell, for example, for E. coli ssb gene, an E. coli ssb gene promoter.

In one embodiment, the SSB protein is an E. coli SSB protein

The nucleic acid vectors of the invention may comprise further additional components. These additional features may be used, for example, to help stabilize transcripts for translation, increase the level of gene expression, and turn on/off gene transcription.

It will be understood by those skilled in the art that the nucleic acid sequences can be operably associated with appropriate control sequences. For example, the nucleic acid sequences can be operably associated with expression control elements, such as transcription/translation control signals, origins of replication, polyadenylation signals, internal ribosome entry sites (IRES), promoters, and/or enhancers, and the like.

In one embodiment, the nucleic acid vector additionally comprises a transcription regulation element. For example, any of the elements described herein may be operably linked to a promoter so that expression can be controlled. In one embodiment, the promoter is a high efficiency promoter

In one embodiment, the promoter is any one of T7, T7lac, Sp6, araBAD, trp, lac, Ptac or pL. The T7 and T7 lac promoters are promoters from the T7 bacteriophage, the latter with a lac operator. Sp6 is a promoter from Sp6 bacteriophage, araBAD is a promoter from the arabinose metabolic operon, trp is a promoter from E. coli tryptophan operon, lac is a promoter from the lac operon and Ptac is a hybrid promoter of the lac and trp promoters and pL is a promoter from the bacteriophage lambda.

### Uses

According to one aspect of the invention, there is provided a use of the nucleic acid vector in the production of a recombinant parvovirus vector particle, optionally a recombinant AAV vector particle, a recombinant BoV vector particle or a recombinant MVM vector particle.

In one embodiment, the nucleic acid sequence encoding the single strand binding protein is on a separate nucleic acid vector to the nucleic acid vector comprising the nucleic acid sequence comprising a parvovirus terminal repeat sequence.

### Methods

According to one aspect of the invention, there is provided a method of propagation and purification of a nucleic acid vector comprising the steps of:

(i) introducing a nucleic acid vector as described herein into a cell

(ii) growing a culture of the cell of step (i)

(iii) harvesting and lysing the cells of step (ii)

(iv) purifying the nucleic acid vector from the lysed cells of step (iii).

In one embodiment, method of the propagation and purification of the nucleic acid vector (plasmid) forms part of a process for recombinant parvovirus vector particle production.

As outlined previously, a common method in the art for producing recombinant parvovirus vector particle, a gene of interest (transgene) is cloned between the terminal repeat sequences of the parvoviral genome. A plasmid carrying nucleic acid sequences of the transgene flanked by parvovirus terminal repeat sequences is commonly referred to as a transfer vector. The transfer vector is then introduced into a cell for propagation and purification. In one embodiment, the method additionally comprises the step of using the nucleic acid vectors described herein, in the cloning of the transgene between the terminal repeat sequences.

If an exogenous nucleic acid sequence encoding the SSB protein is introduced into a prokaryotic cell, it may be desirable to express the nucleic acid sequence comprising the terminal repeat sequences at a different level to the ssb gene. In this case, the nucleic acid sequence encoding the SSB protein may be provided on separate nucleic acid vector with a different origin of replication, to the nucleic acid vector comprising a nucleic acid sequence comprising the terminal repeat sequence. Therefore, in one embodiment, the nucleic acid sequence encoding the single strand binding protein is introduced into the cell in a separate nucleic acid vector to the nucleic acid vector comprising the nucleic acid sequence comprising a parvovirus terminal repeat sequence in step (i).

It will be understood that the embodiments described herein may be applied to all aspects of the invention. Furthermore, all publications, including but not limited to patents and patent applications, cited in this specification are herein incorporated by reference as though fully set forth.

### EXAMPLES

The E. coli ssb gene and a bacterial promoter were cloned into the backbone of the EGFP transfer vector plasmid, pG.AAV2.C.GFP.P2a.fLuc.W6, which contains EGFP downstream of a P_{CMV} promoter flanked by 2 AAV2 ITRs. The effect of the extra copies of ssb gene could then be gauged by the proportion of linearised plasmid following SmaI digest of plasmid preparations. An intact ITR sequence contains a SmaI restriction site. Therefore, if both ITRs are intact, the SmaI digest will result in two fragments. If one SmaI restriction site is lost via ITR deletion, the digest will linearise the plasmid resulting in a single fragment.

### Example 1: Design of the E. Coli ssb Sequence

The E. coli ssb gene coding for single-stranded DNA-binding protein was obtained from GenBank (J01704). This sequence lacked the full native promoter. The coding sequence was synthesised.

Primers ssb-F1 and ssb-R1 from Andreoni et al. (Andreoni et al., (2009) FEBS Letters 584:153-158) were to perform an in-silico PCR against the E. coli genome using the In silico simulation of molecular biology experiments website (http://insilicaehu.eus). The sequence included the full native promoter of ssb gene. The first 209 bp of this sequence was synthesised to obtain the native E. coli ssb promoter.

### Example 2: PCR of ssb and Native Promoter

The ssb coding region and the native promoter were amplified using their respective Gibson assembly primers in which the 3′ end of the native promoter overlapped with the 5′ end of ssb. The PCRs were performed using Q5 High-Fidelity 2X Master Mix (NEB Cat. No. M0492S). The PCR thermal cycling was performed using a Bio-Rad C1000 Touch thermal cycler. The conditions for the reactions were as follows using pUC57.ssb and pUC57.ssb native promoter as template:

| 98° C. | 30 | sec | | 1 cycle |
|---|---|---|---|---|
| 98° C. | 15 | sec | | |
| 58° C. | 15 | sec | {close oversize brace} | 4× |
| 72° C. | 5:30 | min | | |
| 98° C. | 15 | sec | | |
| 64° C. | 15 | sec | {close oversize brace} | 32× |
| 72° C. | 5:30 | min | | |

| | | |
|---|---|---|
| 72° C. | 5:00 | 1 cycle |
| 4° C. | Hold | |

The PCR reactions were subjected to gel electrophoresis on a 0.8% agarose gel containing 1×TAE and 1×SYBR Safe at 80 V for 1 hour. The gel showed that the correct 249 bp native promoter and 702 bp ssb fragments had been amplified. The correct sized PCR products were excised from the gel using a scalpel and the DNA purified using a Qiaquick Gel Extraction kit (Qiagen Cat. No. 28706).

### Example 3: PCR to Join ssb and Native Promoter

PCR was set up to join the ssb and native promoter fragments. The two gel purified fragments with overlapping ends were used in a PCR. The PCRs were performed using Q5 High-Fidelity 2× Master Mix. The PCR thermal cycling was performed using a Bio-Rad C1000 Touch thermal cycler. The conditions for the reactions were the same as the previous PCR.

Following thermal cycling, the PCR reactions were subjected to gel electrophoresis on a 0.8% agarose gel containing 1×TAE and 1×SYBR Safe at 80 V for 1 hour.

The gel showed that the correct 896 bp ssb +native promoter fragment had been amplified. The PCR product was excised from the gel using a scalpel and the DNA purified using a Qiaquick Gel Extraction kit. A ligation was set up containing 0.5 μl pCR-Blunt II-TOPO, 1 μl of salt solution and 4.5 μl of the gel purified PCR product. The ligation was incubated at room temperature for 5 minutes and then 2 μl was used to transform a vial of OneShot TOP10 chemically competent E. coli (Thermo Fisher Cat. No. C404003). The transformed cells were spread on an LB agar plate containing 50 μg/ml Kanamycin and incubated at 37° C. overnight. The following day, colonies were picked from the transformation plate and subcultured on LB agar plates containing 50 μg/ml Kanamycin. The subcultured colonies were grown in 3 ml LB broth cultures containing 50 μg/ml Kanamycin at 37° C. overnight with gentle agitation. The following day the plasmid DNA was extracted from the broth cultures using a QiaPrep Spin Miniprep kit (Qiagen Cat. No. 27106). The concentration of DNA in each of the minipreps was calculated using a Nanodrop and 1 μg of each was digested with EcoRI FD. The digests were incubated at 37° C. for 2 hours and then subjected to gel electrophoresis on a 0.8% agarose gels containing 1×TAE and 1×SYBR Safe at 80 V for 1 hour.

The gel showed that the correct ssb+native promoter fragment had been cloned into pCR-Blunt II-TOPO. These fragments were excised from the gel using a scalpel and the DNA purified using a Qiaquick Gel Extraction kit.

### Example 4: Cloning of ssb+Native Promoter into EGFP Transfer Vector

The EGFP transfer vector plasmid, pG.AAV2.C.GFP.P2a.fLuc.W6, has a unique EcoRI restriction site outside of the transfer vector sequence flanked by the ITRs. The plasmid was digested with EcoRI. The digest was incubated at 37° C. for 2 hours and then subjected to gel electrophoresis on a 0.8% agarose gel containing 1×TAE and 1×SYBR Safe at 80 V for 1 hour.

The linearized plasmid was excised from the gel using a scalpel and the DNA purified using a Qiaquick Gel Extraction kit. The purified fragment was then dephosphorylated with FastAP (Thermo Fisher Cat. No. EF0651). This was then used in a ligation with the gel purified EcoRI digested ssb+native promoter fragment. The ligation reaction contained 2 μl digested transfer vector, 6 μl digested ssb+native promoter fragment, 1 μl 10×ligase buffer and 1 μl T4 DNA ligase. The ligation was incubated overnight at 16° C. in a thermal cycler.

The following day, 2 μl of the ligation was used to transform a vial of OneShot TOP10 chemically competent E. coli. The transformed cells were spread on an LB agar plate containing 50 μg/ml Kanamycin and incubated at 30° C. overnight. The following day, colonies were picked from the transformation plate and subcultured on LB agar plates containing 50 μg/ml Kanamycin. The subcultured colonies were grown in 3 ml LB broth cultures containing 50 μg/ml Kanamycin at 30° C. overnight with gentle agitation. The following day the plasmid DNA was extracted from the broth cultures using a QiaPrep Spin Miniprep kit. The concentration of DNA in each of the minipreps was calculated using a Nanodrop and 1 ug of each was digested with SmaI FD. The digests were incubated at 37° C. for 30 minutes and then subjected to gel electrophoresis on a 0.8% agarose gel containing 1×TAE and 1×SYBR Safe at 80 V for 70 minutes.

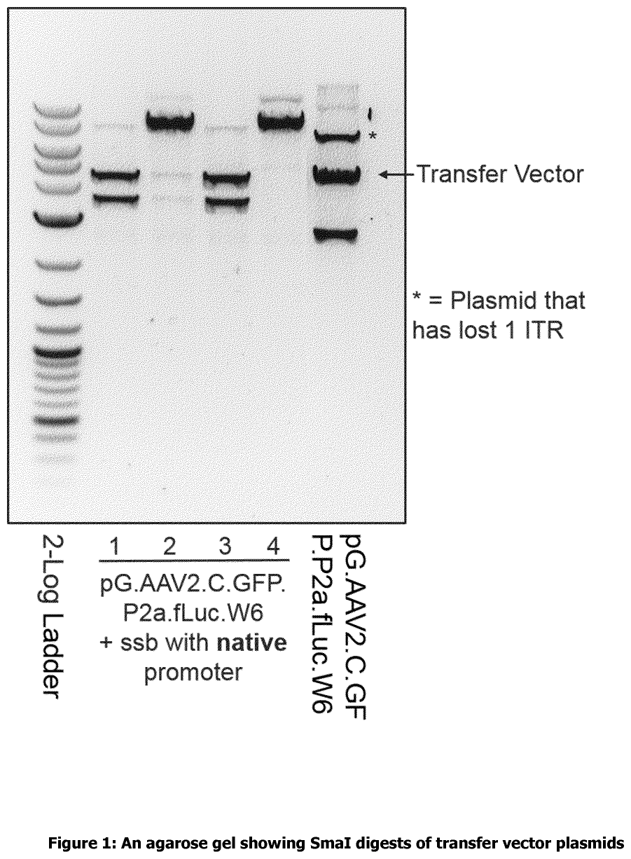

With clones 2 and 4 of FIG. 1, the ssb gene had inserted into a transfer vector plasmid that had already lost 1 ITR. This meant that all the plasmid DNA in these 2 clones were simply linearised. However, clones 1 and 3 contained both ITRs and ssb had inserted into the plasmid backbone. The SmaI digests of these plasmids revealed that the proportion of plasmid that had lost 1 ITR was very low compared to the parental plasmid. It showed that SSB was stabilising the ITRs in these plasmids.

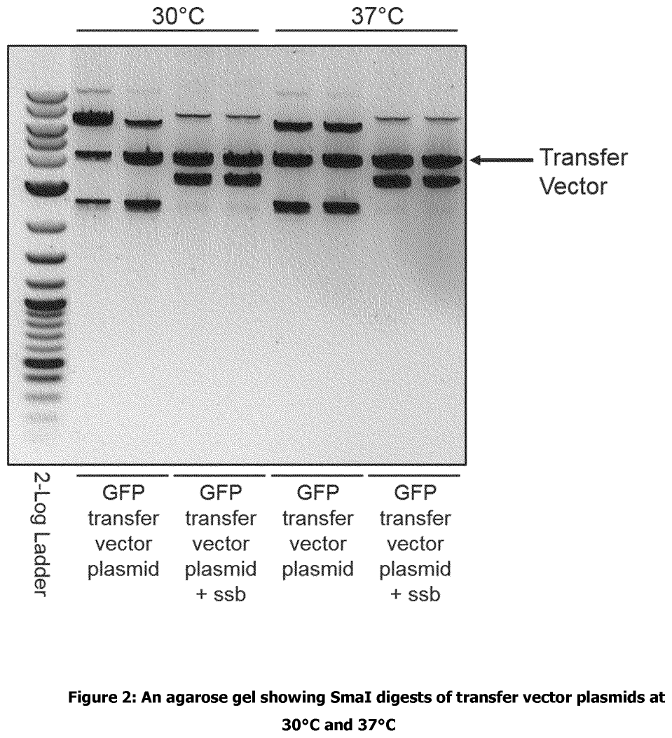

In order to determine whether this effect could still be seen when the E. coli broth cultures were grown at 37° C. Subculture colonies were picked and used to infect LB broth cultures containing 50 μg/ml Kanamycin in duplicate, along with colonies of the original pG.AAV2.C.GFP.P2a.fLuc.W6, that were grown overnight at both 30° C. and 37° C. The following day the plasmid DNA was extracted from the broth cultures using a QiaPrep Spin Miniprep kit. The concentration of DNA in each of the minipreps was calculated using a Nanodrop and 1 ug of each was digested with SmaI FD. The digests were incubated at 37° C. for 30 minutes and then subjected to gel electrophoresis on a 0.8% agarose gels containing 1×TAE and 1×SYBR Safe at 80 V for 70 minutes (FIG. 2).

FIG. 2 showed that at both 30° C. and 37° C., the transfer vector plasmid containing the ssb gene had significantly lower levels of ITR loss, as seen by linearised plasmid, than plasmid that did not contain the ssb gene.

1. A prokaryotic cell comprising a nucleic acid sequence comprising a parvovirus terminal repeat sequence, wherein the prokaryotic cell overexpresses single strand binding protein compared to a prokaryotic cell of a wild-type (WT) strain of the same species.

2. The prokaryotic cell according to claim 1, wherein the cell is a RecA or a functional homologue deficient strain.

3. The prokaryotic cell according to claim 1, wherein the cell is an SbcCD or a functional homologue deficient strain.

4. The prokaryotic cell according to claim 1, wherein the parvovirus is an adeno-associated virus (AAV), a Bocavirus (BoV) or a minute virus of mice (MVM).

5. The prokaryotic cell according to claim 1, comprising an exogenous nucleic acid sequence encoding the single strand binding protein.

6. The prokaryotic cell according to claim 1, wherein the overexpressed single strand binding protein is a variant endogenous to the WT strain of the cell.

7. The prokaryotic cell according to claim 1, wherein the prokaryotic cell is a bacterial cell, optionally of the genus Escherichia, Bacillus, Pseudomonas, Streptomyces, Streptococcus or Vibrio.

8. The cell according to claim 7, wherein the genus is Escherichia, and wherein the cell is Escherichia coli (E. coli).

9. A nucleic acid vector comprising a nucleic acid sequence comprising a parvovirus terminal repeat sequence and a nucleic acid sequence encoding a single strand binding protein.

10. The nucleic acid vector according to claim 9, wherein the parvovirus is AAV, BoV or MVM.

11. The nucleic acid vector according to claim 10, wherein the parvovirus is AAV, and wherein the nucleic acid sequence comprising a parvovirus terminal repeat sequence is derived from AAV1, AAV2, AAV3, AAV4, AAVS, AAV6, AAV7, AAV8, AAV9, AAV10, AAV11, AAV12, AAV13 or combinations thereof.

12. The nucleic acid vector according to claims 9, wherein the single strand binding protein is operably linked to a promoter.

13. The nucleic acid vector according to claim 12, wherein the promoter is a native promoter of the ssb gene.

14. The nucleic acid vector according to claim 12, wherein the promoter is any one of T7, T7lac, Sp6, araBAD, trp, lac, Ptac or pL.

15. The nucleic acid vector according to claim 9, wherein the single strand binding protein is an E. coli single strand binding protein.

16. (canceled)

17. (canceled)

18. A method of propagation and purification of a nucleic acid vector comprising:
(i) introducing a nucleic acid vector of claim 9 into a cell
(ii) growing a culture of the cell of step (i)
(iii)harvesting and lysing the cells of step (ii)
(iv)purifying nucleic acid vector from the lysed cells of step (iii).

19. The method of propagation and purification of a nucleic acid vector according to claim 18, wherein the plasmid is for recombinant parvovirus vector particle production.

20. The method of claim 18, wherein nucleic acid sequence encoding the single strand binding protein is introduced into the cell in a separate nucleic acid vector to the nucleic acid vector comprising the nucleic acid sequence comprising a parvovirus terminal repeat sequence.

21. A method of producing a recombinant parvovirus vector particle, comprising introducing a nucleic acid vector according to claim 9 into a cell and growing a culture of the cell, optionally wherein the recombinant parvovirus vector particle is a recombinant AAV vector particle or recombinant BoV vector particle.

22. The method according to claim 21, wherein the nucleic acid sequence encoding the single strand binding protein is on a separate nucleic acid vector to the nucleic acid vector comprising the nucleic acid sequence comprising a parvovirus terminal repeat sequence.
